(19) United States
(12) Patent Application Publication
(10) Pub. No.: US 2026/0132370 A1
(43) Pub. Date: May 14, 2026

(54) SYSTEM AND APPARATUS OF CELL CULTURING WITH BOTH MASS PRODUCTION AND CUSTOMIZATION

(71) Applicant: CellFabs Inc., Tainan City (TW)

(72) Inventors: Yun-Shu CHIOU, Tainan City (TW); Shang-Chih LIN, Tainan City (TW); Chien-An LU, Tainan City (TW); Chih-Young HUNG, Tainan City (TW); Chien-Chang HUANG, Tainan City (TW)

(73) Assignee: CellFabs Inc., Tainan City (TW)

(21) Appl. No.: 19/237,408

(22) Filed: Jun. 13, 2025

(30) Foreign Application Priority Data
Nov. 12, 2024 (TW) 113143389

(51) Int. Cl. C12M 1/36; C12M 1/00; C12M 1/24; C12M 1/34; C12M 3/00; C12M 3/06

(52) U.S. Cl. CPC C12M 41/48; C12M 23/08; C12M 23/34; C12M 23/48; C12M 27/16; C12M 27/18; C12M 29/18; C12M 41/14; C12M 41/32; C12M 41/34; C12M 41/36; C12M 47/02

(57) ABSTRACT

A system and apparatus of cell culturing with capabilities for both mass production and customization, comprising: a cell culture chamber for culturing a plurality of cell culture flasks, wherein a transfer apparatus is disposed within the cell culture chamber for gripping the cell culture flask; a main track extending from an inlet/outlet of the cell culture chamber to an inspection area, a media exchange area, and a harvesting area, wherein at least one autonomous carrier tray slides along the main track, the autonomous carrier tray being configured to provide placement and retrieval of the cell culture flask by the transfer apparatus; and an intelligent management system configured to execute a cell culture process by controlling the transfer apparatus and the autonomous carrier tray, transporting the cell culture flask to the inspection area for cell inspection, and automatically adjusting the cell culture process based on the cell culture status via artificial intelligence, thereby achieving the efficacy of culturing cells in both a mass production and customized manner.

### BACKGROUND OF THE DISCLOSURE

### 1. Field of the Disclosure

The present invention relates to an automated cell culture factory, and more particularly to a system that integrates a large-scale cell culture chamber, an inspection area, a media exchange area, and a harvesting area, utilizing an intelligent management system to achieve both mass production and customized cell culturing capabilities.

### 2. Description of the Prior Art

Cell culture is a core component in immunotherapy and regenerative medicine; however, current global cell culture technologies face numerous difficulties and challenges. First, cell culture is typically highly customized according to the needs of specific medical cases and relies on manual operations conducted within cell laboratories. This process is vulnerable to environmental interference, which results in a high risk of failure. Factors such as ambient temperature, humidity, carbon dioxide concentration, the stability of the culture medium composition, and bacterial contamination can all lead to instability in the culturing process, and in severe cases, cause complete failure. Such failures not only increase costs but, more critically, may delay medical treatment and adversely impact patient outcomes.

The overall cell culture cycle is relatively long, with each batch typically requiring around two weeks. During this period, the condition of the cultured cells must be continuously monitored and adjusted, relying heavily on daily observations and real-time responses from researchers. This demands considerable manpower and significantly drives up costs, creating a major bottleneck for cost control in regenerative medicine. Due to the high technical costs, cell culture technologies have not been widely adopted in standard medical settings, limiting the societal benefits of regenerative medicine. Existing cell culture equipment designs—whether domestic or international—focus primarily on “alleviating the burden on researchers” rather than fully replacing manual labor. For instance, robotic arms may be used to perform repetitive tasks, but these are partial solutions that fail to manage the culturing process from a systems perspective. While they can improve production capacity, they fall short in addressing customization needs, thus failing to reconcile the high-volume demands of a cell factory with the personalized needs of a laboratory. Consequently, current automated cell culture systems do not fundamentally resolve the issues of high labor demand and weak risk control, as they lack systematic integration. The result is an ongoing dependence on human oversight, which raises operational costs and failure risks, ultimately hindering the scalability and widespread adoption of regenerative medicine.

In view of the foregoing, the inventor, drawing on years of experience in the development and design of related products, has carefully conceived and evaluated this invention, which offers practical and effective solutions to the aforementioned challenges.

### SUMMARY OF THE DISCLOSURE

The technical problem to be solved by the present invention is to provide a system and apparatus of cell culturing with capabilities for both mass production and customization, addressing the deficiencies present in existing technologies.

A cell culture chamber is provided for culturing a plurality of cell culture flasks, wherein a transfer apparatus is arranged inside the cell culture chamber for gripping the cell culture flask and moving it to the entry/exit of the cell culture chamber. An inspection area is configured to monitor the culturing status of the cell culture flask through optical microscopy. A media exchange area is configured to perform culture medium replacement for the cell culture flask using at least one media exchange syringe. A harvesting area is configured to transfer the liquid within the cell culture flask into a harvesting bottle, and the harvesting bottle separates the cells from the culture medium via centrifugation. A main track extends from the entry/exit of the cell culture chamber to the inspection area, media exchange area, and harvesting area. At least one autonomous carrier tray slides along the main track, and serves as a platform for the transfer apparatus to place and retrieve the cell culture flask, autonomously traveling among the inspection area, media exchange area, and harvesting area. An intelligent management system is provided to generate identification markers and define culturing workflows for the cell culture flask based on the type of cell. It executes the culturing workflows by controlling the transfer apparatus and autonomous carrier tray, transporting the cell culture flask to the inspection area for cellular inspection, and automatically adjusting the culturing workflow based on the culturing status using artificial intelligence.

The media exchange area includes a first supply track, which delivers the media exchange syringe for use in the media exchange area. The media exchange area further includes at least one culture medium container and one waste liquid container. The waste liquid container receives spent culture medium extracted from the cell culture flask by the media exchange syringe, while the culture medium container provides fresh medium to the media exchange syringe for injection into the cell culture flask.

The harvesting area includes a second supply track that delivers the harvesting bottle for use in the harvesting area. The harvesting area also employs at least one harvesting syringe to extract the culture medium from the cell culture flask and to fill it into the harvesting bottle.

The transfer apparatus comprises at least one transfer track, a rotation shaft, a translation shaft, and a gripping unit. The transfer track is fixed inside the cell culture chamber, with one end positioned at the entry/exit of the cell culture chamber. The rotation shaft is movably mounted on the transfer track, the translation shaft is movably mounted on the rotation shaft, and the gripping unit is movably mounted on the translation shaft. The gripping unit is configured to grip the cell culture flask.

The transfer apparatus is equipped with an imaging lens at the gripping unit to capture the color of the culture medium in the cell culture flask. Through artificial intelligence, the color of the culture medium is analyzed, and when deviations from the acceptable range are detected, the system automatically adjusts the culturing workflow and initiates inspection, media exchange, harvesting, or discard procedures in real time.

The transfer apparatus is further equipped with a marker verifier at the gripping unit, which is used to identify the cell culture flask's recognition marker. The actual culturing process of the cell culture flask is recorded into the intelligent management system to create a culture log. A large volume of such culture logs is collected to train the artificial intelligence within the intelligent management system, thereby optimizing adjustments to the temperature, humidity, and carbon dioxide concentration in the cell culture chamber, as well as the media exchange interval and medium composition for the cell culture flask.

The intelligent management system communicates with multiple mobile devices via a wireless network. Each mobile device accesses the cell information, cell data, culture log, and real-time monitoring images of the cell culture flask based on its identification tag.

The cell culture chamber is partitioned into multiple storage compartments, each configured to hold a cell culture flask. Each storage compartment is equipped with a shaker, which is controlled by the intelligent management system to perform scheduled shaking of the cell culture flask.

The cell culture flask is formed with multiple helical recesses on the bottle body. When shaken by the shaker, the culture medium inside the cell culture flask circulates upward in a vortex along the helical recesses.

The shaker includes a positioning disc for placing the cell culture flask, and the positioning disc is provided with multiple protruding pegs. The bottom of the cell culture flask is recessed to form multiple positioning recesses, which engage with the protruding pegs to fix the position of the cell culture flask.

The primary objective of the present invention is that the cell culture chamber not only provides a suitable environment for culturing but also enables the transfer apparatus to automatically grip the cell culture flask, allowing personnel to avoid entering the cell culture chamber. In combination with the main track, the autonomous carrier tray transports the cell culture flask and, coordinated by the intelligent management system, autonomously operates both the transfer apparatus and the autonomous carrier tray to deliver the cell culture flask to the inspection area for cell inspection. The system further transports the cell culture flask to the media exchange area for media replacement and to the harvesting area for cell harvesting. Artificial intelligence is used to automatically adjust the cell culture workflow based on the current cell status. This setup achieves the dual function of fully automated mass production and fully automated customization in cell culture. Moreover, data related to cell culture success and failure are stored in the intelligent management system, enabling data analysis and machine learning to automatically optimize the cell culture workflow.

Other objects, advantages, and novel features of the present creation will become more apparent from the following detailed description and the accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is an operational flowchart of the cell culture factory according to the present invention;

FIG. 2 is a system block diagram of the cell culture factory according to the present invention;

FIG. 3 is a schematic layout diagram of the areas within the cell culture factory according to the present invention;

FIG. 4 is a perspective view of the cell culture chamber according to the present invention;

FIG. 5 is a front schematic view of the cell culture chamber according to the present invention;

FIG. 6 is a perspective view of the autonomous carrier tray according to the present invention; and

FIG. 7 is a perspective view of the shaker and the cell culture flask according to the present invention.

### DETAILED DESCRIPTION

In order to enable the esteemed examiner to further understand and recognize the objectives, characteristics, and advantages of the present invention, a detailed description is provided below with reference to the accompanying drawings.

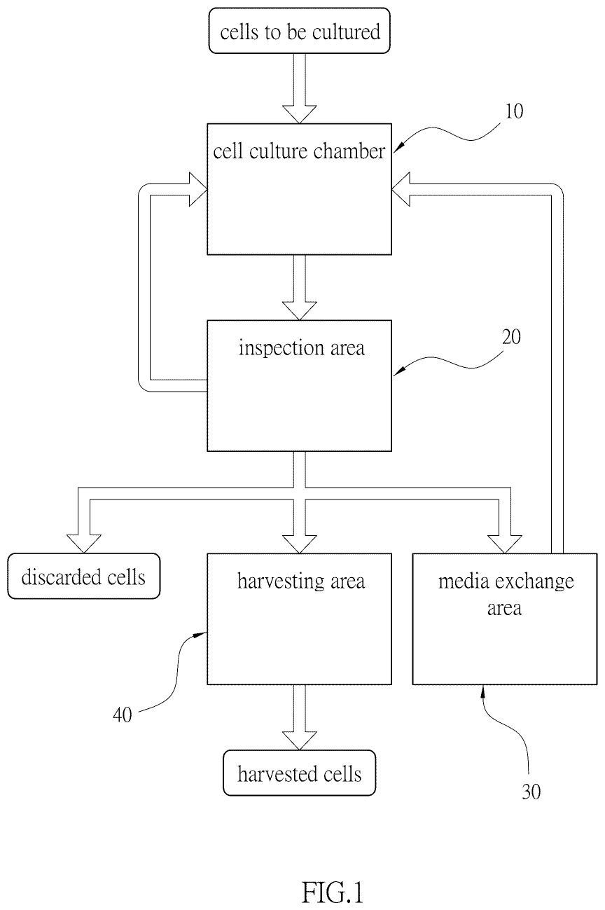

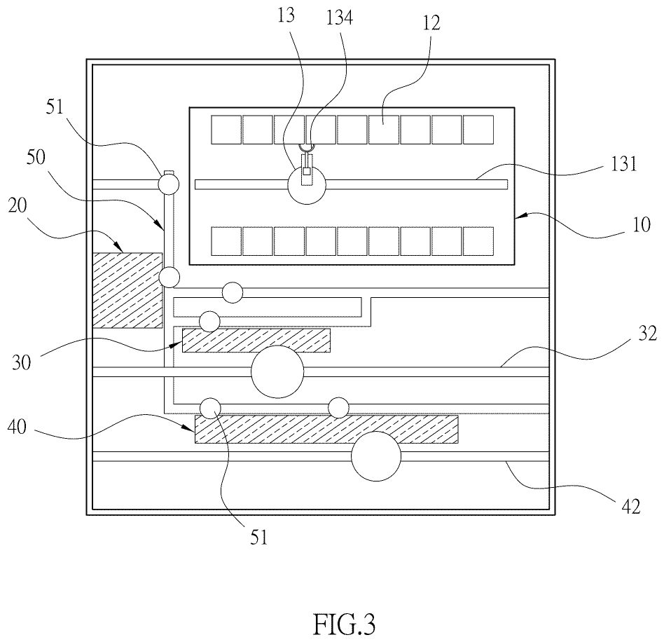

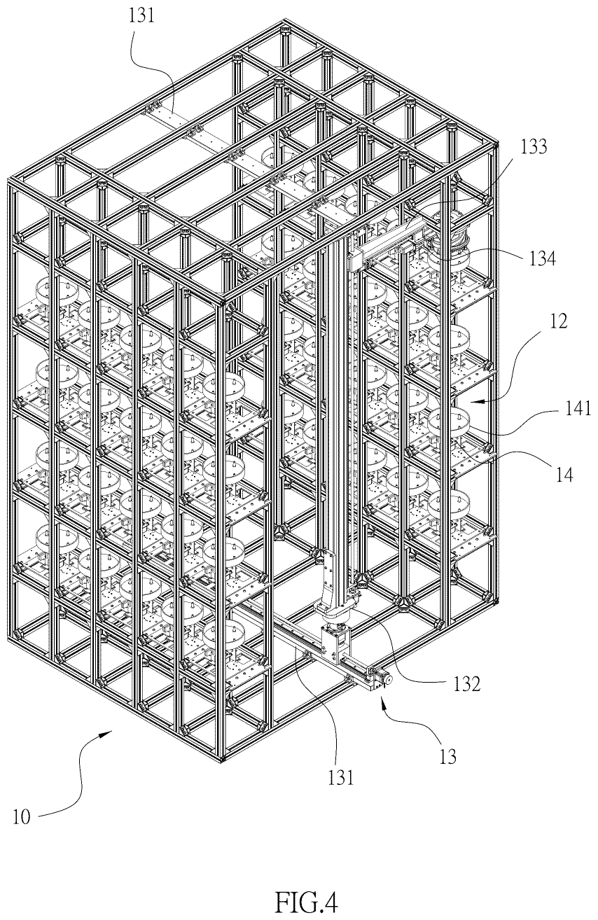

Referring to FIGS. 1 to 4, a system and apparatus of cell culturing with capabilities for both mass production and customization is disclosed. The system includes a cell culture chamber 10, an inspection area 20, a media exchange area 30, a harvesting area 40, a main track 50, and an intelligent management system 60. The cell culture chamber 10 is configured for culturing a plurality of cell culture flasks 11, and it is partitioned into multiple storage compartments 12, each of which is used to store a cell culture flask 11. The number of storage compartments 12 can be adjusted according to actual requirements to meet mass production needs. A transfer apparatus 13 is provided inside the cell culture chamber 10 for gripping and transferring a cell culture flask 11 to and from the entry/exit point of the chamber. This transfer minimizes exposure to external air, thereby maintaining stable temperature, humidity, and carbon dioxide concentration within the cell culture chamber 10.

The inspection area 20 utilizes optical microscopy to detect the culturing status of the cell culture flasks 11. This inspection method can be an Automated Optical Inspection (AOI) system that captures optical images for high-speed, high-accuracy computer vision analysis, allowing cell counts to be determined from within the cell culture flask 11. Based on the inspection results, each cell culture flask 11 is directed to the next workstation. In other words, every cell culture flask 11 transferred out of the cell culture chamber 10 is first sent to the inspection area 20 for cell condition assessment.

The media exchange area 30 performs culture medium replacement on the cell culture flasks 11 using at least one media exchange syringe 31. The media exchange area 30 includes a first supply track 32 that delivers the media exchange syringe 31 for use in the media exchange area 30. Used media exchange syringes 31 are also discharged via the first supply track 32. Additionally, the media exchange area 30 includes at least one culture medium container 33 and one waste liquid container 34. The waste liquid container 34 collects waste medium extracted from the cell culture flask 11 via the media exchange syringe 31, while the culture medium container 33 provides fresh medium for the media exchange syringe 31 to inject into the cell culture flask 11. Multiple culture medium containers 33 can be loaded with culture media of various compositions or concentrations, enabling each media exchange syringe 31 to draw specific portions and blend them into an optimal formulation tailored for customized cell culture.

A harvesting area 40 is configured to transfer the liquid contents from the cell culture flask 11 into a harvesting bottle 41. The harvesting bottle 41 separates the cells from the culture medium via centrifugation, thereby collecting the harvested cells. The harvesting area 40 includes a second supply track 42, which delivers the harvesting bottle 41 for use within the harvesting area 40. The harvesting area 40 utilizes at least one harvesting syringe 43 to extract the culture medium from the cell culture flask 11 and fill it into the harvesting bottle 41.

A main track 50 extends from the entrance/exit of the cell culture chamber 10 to the inspection area 20, the media exchange area 30, and the harvesting area 40. At least one autonomous carrier tray 51 travels along the main track 50, driven by a motor, and autonomously moves to designated positions. The autonomous carrier tray 51 facilitates the placement and retrieval of the cell culture flask 11 by the transfer apparatus 13 and autonomously transports the flask between the inspection area 20, the media exchange area 30, and the harvesting area 40. Upon completing inspection or media exchange, the autonomous carrier tray 51 returns the cell culture flask 11 to the entrance/exit of the cell culture chamber 10, where the transfer apparatus 13 grips and places it back into the corresponding storage compartment 12, thereby completing a cycle of periodic inspection and media exchange for the cell culture flask 11. Similarly, when the inspection area 20 determines that cell harvesting is appropriate, the autonomous carrier tray 51 transports the cell culture flask 11 to the harvesting area 40.

An intelligent management system 60 provides identification markers and cell culture protocols for the cell culture flask 11 based on cell type. Each new cell culture flask 11 is assigned a unique identification marker, which may be a chip, QR code, NFC, or an equivalent identification method. Once the cell culture flask 11 is placed within the cell culture chamber 10, the intelligent management system 60 autonomously executes the cell culture process, controlling the transfer apparatus 13 and the autonomous carrier tray 51 to transport the cell culture flask 11 to the inspection area 20 for cell inspection, the media exchange area 30 for media replacement, and the harvesting area 40 for cell harvesting. The system utilizes artificial intelligence to automatically adjust the cell culture process based on the cell culture status, thereby achieving fully automated mass production and customized cell culture. Data on successful and failed cell cultures are stored within the intelligent management system 60, enabling process optimization through data analysis and machine learning.

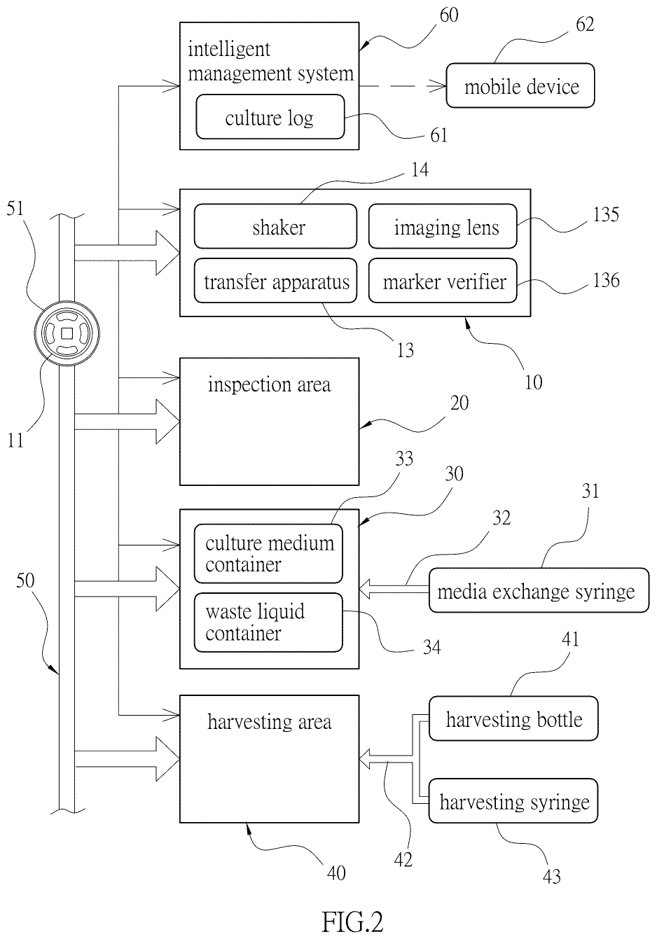

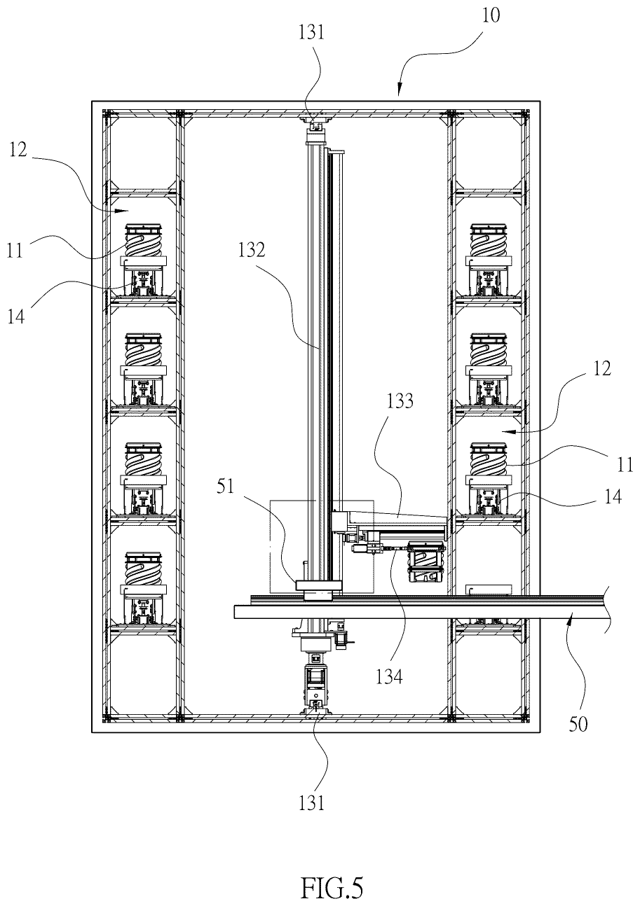

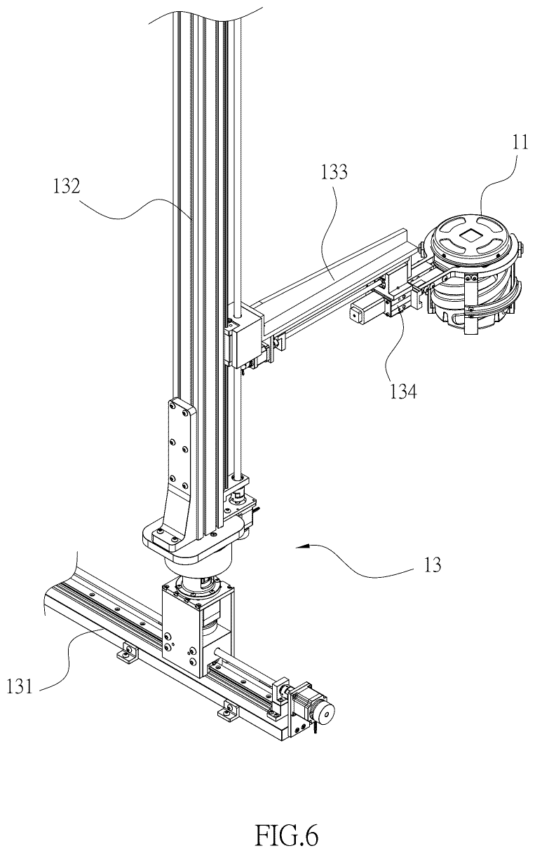

Further elaborating with reference to FIGS. 2, 5, and 6, the transfer apparatus 13 comprises at least one transfer track 131, a rotation shaft 132, a translation shaft 133, and a gripping unit 134. The transfer track 131 is fixed within the cell culture chamber 10, allowing the storage compartments 12 to be arrayed on both sides of the transfer track 131, with one end positioned at the entrance/exit of the cell culture chamber 10. The rotation shaft 132 is movably mounted on the transfer track 131 and can reciprocate along it, as well as perform axial rotation. The translation shaft 133 is movably mounted on the rotation shaft 132 and can reciprocate along it. The gripping unit 134 is movably mounted on the translation shaft 133 and can move back and forth along it. When the gripping unit 134 approaches a storage compartment 12, it grips the cell culture flask 11. Through the transfer apparatus 13's gripping and placement of the cell culture flask 11, automated management is achieved.

Referring also to FIG. 2, the transfer apparatus 13 is equipped with an imaging lens 135 at the gripping unit 134. The imaging lens 135 captures the color of the culture medium inside the cell culture flask 11. Based on artificial intelligence analysis, when the color of the culture medium deviates beyond acceptable thresholds, the system automatically adjusts the cell culture process and promptly performs AOI inspection, media replacement, cell harvesting, or waste cell disposal. Furthermore, the transfer apparatus 13 is equipped with a marker verifier 136 at the gripping unit 134, which identifies the marker of the cell culture flask 11. The actual cell culture process of the cell culture flask 11 is recorded within the intelligent management system 60 as a culture log 61, and a large volume of these culture log 61 entries are used to train the artificial intelligence module of the intelligent management system 60. This optimizes the regulation of temperature, humidity, and CO₂ concentration in the cell culture chamber 10, as well as the frequency and composition of media exchanges for the cell culture flask 11. The intelligent management system 60 communicates wirelessly with multiple mobile device 62, which retrieve cell information, cell data, the culture log, and real-time monitoring screens based on the identification of the cell culture flask 11, enabling personnel to track cell culture progress in real time.

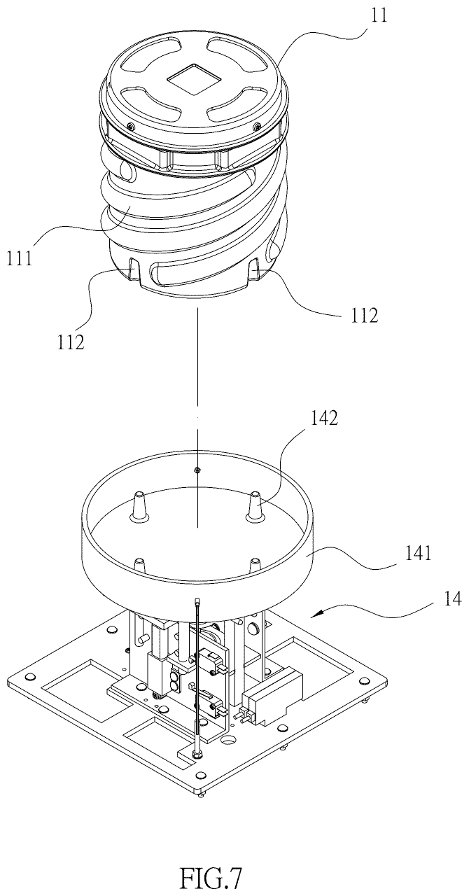

Referring also to FIGS. 2, 5, and 7, the storage compartment 12 defined within the cell culture chamber 10 is configured to hold the cell culture flask 11. Each storage compartment 12 is equipped with a shaker 14, which is controlled by the intelligent management system 60 to perform shaking actions for designated durations. This prevents cell aggregation due to static placement and enhances cell interaction with CO₂, thereby effectively increasing cell viability. The cell culture flask 11 is formed with multiple helical recess 111 on its body, which facilitate upward vortex flow of the culture medium along the helical recess 111 when agitated by the shaker 14, thereby improving uniform mixing. The shaker 14 includes a positioning disc 141 for supporting the cell culture flask 11, and the positioning disc 141 is provided with multiple protruding peg 142. The bottom of the cell culture flask 11 has multiple positioning recess 112, and the engagement between the positioning recess 112 and the protruding peg 142 secures the cell culture flask 11 in place, preventing it from dislodging during operation.

It is to be understood that the above-described embodiments are merely preferred examples of the present invention, and should not be construed as limiting the scope of implementation. Any equivalent modifications or alterations made according to the claims of the present invention are intended to fall within the scope of the present disclosure.

What is claimed is:

1. A system and apparatus of cell culturing with capabilities for both mass production and customization, comprising:
a cell culture chamber, configured to culture a plurality of cell culture flask, the cell culture chamber being equipped with a transfer apparatus, wherein the transfer apparatus is used to grip and move the cell culture flask to and from the entry/exit of the cell culture chamber;
an inspection area, configured to determine the culture status of the cell culture flask via optical microscopy;
a media exchange area, configured to replace the culture medium of the cell culture flask through at least one media exchange syringe;
a harvesting area, configured to transfer the liquid content from the cell culture flask into a harvesting bottle, wherein the harvesting bottle separates cells from the culture medium through centrifugation;
a main track, extending from the entry/exit of the cell culture chamber to the inspection area, media exchange area, and harvesting area, wherein at least one autonomous carrier tray slides along the main track, the autonomous carrier tray enabling the transfer apparatus to place and retrieve the cell culture flask, and autonomously traveling among the inspection area, media exchange area, and harvesting area;
an intelligent management system, configured to generate identification markers and cell culture workflows for the cell culture flask based on cell type, execute the cell culture process, and control the transfer apparatus and autonomous carrier tray to transport the cell culture flask to the inspection area for cell inspection, and to automatically adjust the cell culture workflow using artificial intelligence based on the cell culture status.

2. The system and apparatus of cell culturing with capabilities for both mass production and customization as claimed in claim 1, wherein the media exchange area comprises a first supply track, the first supply track being configured to transport the media exchange syringe to the media exchange area for use, and the media exchange area further comprising at least one culture medium container and one waste liquid container, wherein the waste liquid container is used to receive waste culture medium extracted from the cell culture flask by the media exchange syringe, and the culture medium container supplies fresh culture medium to the media exchange syringe for injection into the cell culture flask.

3. The system and apparatus of cell culturing with capabilities for both mass production and customization as claimed in claim 1, wherein the harvesting area comprises a second supply track, the second supply track being configured to transport the harvesting bottle to the harvesting area for use, and the harvesting area uses at least one harvesting syringe to extract culture medium from the cell culture flask and fill the culture medium into the harvesting bottle.

4. The system and apparatus of cell culturing with capabilities for both mass production and customization as claimed in claim 1, wherein the transfer apparatus comprises at least one transfer track, a rotation shaft, a translation shaft, and a gripping unit, wherein the transfer track is fixed within the cell culture chamber and has one end positioned at the entry/exit of the cell culture chamber, the rotation shaft is movably disposed along the transfer track, the translation shaft is movably disposed along the rotation shaft, and the gripping unit is movably disposed along the translation shaft and configured to grip the cell culture flask.

5. The system and apparatus of cell culturing with capabilities for both mass production and customization as claimed in claim 4, wherein the transfer apparatus is equipped with an imaging lens at the gripping unit, the imaging lens being used to capture the color of the culture medium inside the cell culture flask, and artificial intelligence is used to determine if the color exceeds a reasonable range, thereby automatically adjusting the cell culture process and immediately executing inspection, media exchange, harvesting, or disposal procedures.

6. The system and apparatus of cell culturing with capabilities for both mass production and customization as claimed in claim 5, wherein the transfer apparatus is equipped with a marker verifier at the gripping unit, the marker verifier being configured to identify the identification mark of the cell culture flask, and to establish a culture log in the intelligent management system based on the actual cell culture process of the cell culture flask, whereby a large amount of the culture log is collected to train the artificial intelligence of the intelligent management system, thereby optimizing the adjustment of temperature, humidity, and carbon dioxide concentration in the cell culture chamber, as well as the media exchange interval and culture medium composition for the cell culture flask.

7. The system and apparatus of cell culturing with capabilities for both mass production and customization as claimed in claim 6, wherein the intelligent management system communicates via a wireless network with a plurality of mobile devices, each mobile device being configured to acquire cell information, cell data, culture log, and real-time monitoring images of the cell culture flask based on its identification mark.

8. The system and apparatus of cell culturing with capabilities for both mass production and customization as claimed in claim 1, wherein the cell culture chamber is partitioned into a plurality of storage compartments, each storage compartment being used for placing a cell culture flask, and each storage compartment is equipped with a shaker controlled by the intelligent management system to perform designated shaking operations on the cell culture flask.

9. The system and apparatus of cell culturing with capabilities for both mass production and customization as claimed in claim 8, wherein the cell culture flask is formed with a plurality of helical recesses on its bottle body, and during operation of the shaker, the culture medium inside the cell culture flask flows upward in a vortex along the helical recesses.

10. The system and apparatus of cell culturing with capabilities for both mass production and customization as claimed in claim 8, wherein the shaker comprises a positioning disc for supporting the cell culture flask, the positioning disc being provided with a plurality of protruding pegs, and the bottom of the cell culture flask is recessed with a plurality of positioning recesses, the engagement of the positioning recesses with the protruding pegs securing the cell culture flask in place.
